(19) United States
(12) Patent Application Publication
(10) Pub. No.: US 2024/0158511 A1
(43) Pub. Date: May 16, 2024

(54) Humanized mAb107

(71) Applicant: The General Hospital Corporation, Bosdton, MA (US)

(72) Inventor: M. Amin Arnaout, Chestnut Hill, MA (US)

(21) Appl. No.: 18/283,927

(22) PCT Filed: Mar. 25, 2022
(86) PCT No.: PCT/US22/21950

§ 371 Date: Sep. 25, 2023

Related U.S. Application Data
(60) Provisional application No. 63/166,711, filed on Mar. 26, 2021.

(51) Int. Cl. C07K 16/28; A61K 39/00

(52) U.S. Cl. CPC C07K 16/2845; A61K 2039/505; A61K 2039/545; C07K 2317/24; C07K 2317/565; C07K 2317/622

(57) ABSTRACT

Described herein are humanized antibodies that bind to Leukocyte integrin CD11b/CD18 (CD11b, αMβ2, CR3) with enhanced affinity, and methods of use thereof.

### CLAIM OF PRIORITY

This application claims the benefit of U.S. Provisional Patent Application Ser. No. 63/166,711, filed on Mar. 26, 2021. The entire contents of the foregoing are hereby incorporated by reference.

### FEDERALLY SPONSORED RESEARCH OR DEVELOPMENT

This invention was made with Government support under Grant No. DK48549 awarded by the National Institutes of Health. The Government has certain rights in the invention.

### STATEMENT REGARDING SEQUENCE LISTING

This document contains a Sequence Listing that has been submitted electronically as an ASCII text file named sequencelisting. The ASCII text file, created on Mar. 24, 2022, is 21.4 kilobytes in size. The material in the ASCII text file is hereby incorporated by reference in its entirety.

### TECHNICAL FIELD

Described herein are humanized antibodies that bind to Leukocyte integrin CD11b/CD18 (CD11b, αMβ2, CR3) with enhanced affinity, and methods of use thereof.

### BACKGROUND

Leukocyte integrin CD11b/CD18 (also known as CD11b, αMβ2, CR3, referred to herein as CD11b) is the archetypal innate immune receptor expressed on innate immune cells. CD11b binds more than 40 ligands, thus mediating leukocyte adhesion-dependent functions including homing, phagocytosis, adhesion-dependent superoxide generation, proteolytic enzyme and cytokine production, antibody-dependent cytolytic activity, and enhancing adaptive immunity (Arnaout, F1000Res. 2016 Oct. 4; 5:F1000 Faculty Rev-2433; van den Elsen et al., Science (2011) 332:608-1; Bajic et al., Proc Natl Acad Sci USA. (2013) 110:16426-31). Binding of physiologic ligands such as ICAM-1, complement iC3b and fibrinogen to CD11b induces activating tertiary and quaternary changes in CD11b leading to proadhesive cell signaling. It is now established that current anti-integrin agents that stabilize this activated proinflammatory state can cause serious adverse outcomes in humans that were not predicted from the rodent studies (Raab-Westphal et al., Cancers (Basel). 2017 Aug. 23; 9(9):110).

### SUMMARY

Provided herein are antibodies or antigen-binding fragments thereof that bind to CD11b, and methods of use thereof. In some embodiments, the antibodies or antigen-binding fragments thereof comprise an amino acid sequence that comprises the following complementarity determining regions (CDR):

1)
(SEQ ID NO: 5)
CDR 1 of the VH of SEQ ID NO: 1, e.g., GFNIKD;
2)
(SEQ ID NO: 8)
CDR 1 of the VL of SEQ ID NO: 2, e.g.,
SQNLLYSSNQKNY);
3)
(SEQ ID NO: 6)
CDR 2 of the VH of SEQ ID NO: 1, e.g., PADDKTK;
4)
(SEQ ID NO: 9)
CDR 2 of the VL of SEQ ID NO: 2, e.g.,
WASTRESGVPDR;
5)
(SEQ ID NO: 7)
CDR3 of the VH of SEQ ID NO: 1, e.g., GHYGYDGYA;
and
6)
(SEQ ID NO: 10)
CDR 3 of the VL of SEQ ID NO: 2, e.g., YYSYPL.

In some embodiments, the antibodies or antigen-binding fragments thereof comprise a heavy chain variable region comprising VH CDRs 1, 2, 3, and a light chain variable region comprising VL CDRs 1, 2, 3, wherein the VH CDRs 1, 2, 3 are identical to complementarity determining regions in SEQ ID NO: 1, and the VL CDRs 1, 2, 3 are identical to complementary determining regions in SEQ ID NO: 2.

In some embodiments, the antibodies or antigen-binding fragments thereof comprise an amino acid sequence that comprises an amino acid sequence at least 95% identical to an amino acid sequence selected from the group consisting of:

(SEQ ID NO: 3)
QVQLVQSGAEVKKPGASVKVSCKPSGFNIKDIYMQWVRQAPGQRLEWIGR
IDPAdDKTKYDPKFQGRATITADTSASTAYLELSSLRSEDTAVYYCASEG
HYGYdGYAMDYWGQGTTVTVSSASTKGPSVFPLAPCSRSTSESTAALGCL
VKDYFPEPVTVSWNSGALTSGVHTFPAVLQSSGLYSLSSVVTPSSSLGTK
TYTCNVDHKPSNTKVDKR;
(heavy chain, SEQ ID NO: 17)
QVQLVQSGAEVKKPGASVKVSCKPSGFNIKDIYMQWVRQAPGQRLEWIGR
IDPAdDKTKYDPKFQGRATITADTSASTAYLELSSLRSEDTAVYYCASEG
HYGYDGYAMDYWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSC;
(heavy chain, SEQ ID NO: 13)
QVQLVQSGAEVKKPGASVKVSCKPSGFNIKDIYMQWVRQAPGQRLEWIGR
IDPADDKTKYDPKFQGRATITADTSASTAYLELSSLRSEDTAVYYCASEG
HYGYDGYAMDYWGQGTTVTVSSASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK,
and
(light chain, SEQ ID NO: 4)
DIVMTQSPDSLAVSLGERATINCKSSQNLLYSSNQKNYLAWYQQKPGQPP
KLLIYWASTRESGVPDRFSGSGSGTDFTLTISSLQAEDVAVYYCQQYYSY
PLTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC,

provided that the complementarity-determining regions of the sequence are not altered.

In some embodiments, the antibodies or antigen-binding fragments thereof comprise a single chain variable fragment (scFv). In some embodiments, the antibodies or antigen-binding fragments thereof comprise SEQ ID NO: 15.

Further, provided herein are compositions comprising an antibody or antigen-binding fragment thereof as described herein, and a pharmaceutically acceptable carrier

Also provided herein are methods for ameliorating a pathology associated with ischemia reperfusion injury in a subject. The methods include administering to the subject a composition comprising a therapeutically effective amount of an antibody or antigen-binding fragment thereof as described herein.

In some embodiments, the pathology is post-ischemic renal fibrosis.

In some embodiments, the pathology is a kidney fibroinflammatory disease.

In some embodiments, the pathology is pulmonary fibrosis.

In some embodiments, the pathology is post-myocardial infarction left ventricular adverse remodeling.

In some embodiments, the composition is administered to the subject within about 5 hours after the ischemia reperfusion injury.

In some embodiments, the composition is administered to the subject within about 2 hours after the ischemia reperfusion injury.

Also provided herein are methods for treating a subject having or at risk of developing a disorder associated with ischemia reperfusion injury in an organ. The methods include administering to the subject a composition comprising a therapeutically effective amount of an antibody or antigen-binding fragment thereof as described herein.

In some embodiments, the organ is a kidney. In some embodiments, the disorder is acute kidney injury.

In some embodiments, the organ is a heart. In some embodiments, the disorder is acute coronary syndrome. In some embodiments, the disorder is acute myocardial infarction (I).

In some embodiments, the organ is a lung.

In some embodiments, the composition is administered to the subject within about 5 hours after the ischemia reperfusion injury.

In some embodiments, the composition is administered to the subject within about 2 hours after the ischemia reperfusion injury.

Additionally, provided herein are methods for providing an organ for transplantation. The methods include administering to an organ donor a composition comprising an antibody or antigen-binding fragment thereof as described herein; and harvesting the organ from the organ donor.

In some embodiments, the organ is a kidney, a heart, or a lung.

Also provided herein are methods for reducing delayed graft function following organ transplantation. The methods include administering to an organ recipient a therapeutically effective amount of composition comprising an antibody or antigen-binding fragment thereof as described herein prior to transplantation of the organ to the recipient or within a day of the transplantation, thereby reducing delayed graft function following organ transplantation.

In some embodiments, the organ is a kidney, a heart, or a lung.

Further, provided herein are methods for treating an organ prior to transplantation into a recipient. The methods include contacting the organ with a composition comprising an antibody or antigen-binding fragment thereof as described herein.

In some embodiments, the organ is a kidney, a heart, or a lung.

In some embodiments, the contacting step comprises perfusing the organ with the composition comprising a polypeptide or antibody that immunospecifically binds the epitope recognized by mab107.

Additionally, provided herein are methods for treating a subject having an autoimmune disease comprising administering to the subject a therapeutically effective amount of composition comprising an antibody or antigen-binding fragment thereof as described herein.

In some embodiments, the autoimmune disease is cytoplasmic antineutrophil cytoplasmic antibodies (cANCA)-associated vasculitis.

Further provided herein are methods for treating a subject having diabetic nephropathy comprising administering to the subject a therapeutically effective amount of composition comprising an antibody or antigen-binding fragment thereof as described herein.

Also provided herein are methods for ameliorating a pathology associated with chemotherapy in a subject comprising administering to the subject a therapeutically effective amount of composition comprising an antibody or antigen-binding fragment as described herein. In some embodiments, the pathology is Adriamycin nephropathy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Coomassie stain of an SDS-PAGE showing migration of mouse mAb107 and humanized IgG4κ mAb107, mutated to carry a novel Asn²⁵⁷/Asp substitution in its CDR2 heavy chain, under unreducing (left panel) and reducing (right panel) conditions.

FIG. 2. Coomassie stain of an SDS-PAGE showing migration of humanized IgG4κ mAb107 containing wild-type Ser 228 (S) or Pro 228 (P) in the core hinge region of IgG4 under reducing (left) panel of unreduced (right panel) conditions. This substitution reduced the band corresponding to the known Fab arm exchange (which results in functionally monovalent, bispecific antibodies with unknown specificity and hence, potentially, reduced therapeutic efficacy (Silva et al., J Biol Chem. 2015 Feb. 27; 290(9):5462-9). MW standards (in kDa) are shown in lane 1 of the stained gel.

### DETAILED DESCRIPTION

The below paragraphs provide certain definitions in order to provide a clearer and consistent understanding of the specification and claims.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration/Administered: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Animal: as used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities.

Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Humanized: as is known in the art, the term “humanized” is commonly used to refer to antibodies (or antibody components) whose amino acid sequence includes VH and VL region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C^{H}3, and, optionally, a C_{H}4 region of a heavy chain constant region.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Prevent or prevention: as used herein when used in connection with the occurrence of a disease, disorder, and/or condition, refers to reducing the risk of developing the disease, disorder and/or condition and/or to delaying onset and/or severity of one or more characteristics or symptoms of the disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder, or condition is observed in a population susceptible to the disease, disorder, or condition.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Therapeutically Effective Amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2^{nd} ed., Cold Spring Harbor Laboratory Press, Cold Spring Harbor, N.Y. (1989)), which is incorporated herein by reference for any purpose.

### Antibodies of the Disclosure

Antibodies of the disclosure include humanized forms of mAb107, as described in the examples. In some embodiments, the antibodies of the disclosure are anti-CD11b antibodies, or fragments thereof. In some embodiments, the antibodies of the disclosure are recombinant antibodies. In some embodiments, the anti-CD11b antibodies of the disclosure includes 1, 2, or 3 heavy chain CDR sequences that are or include a sequence of SEQ ID NOs: 5, 6, and/or 7. In some embodiments, the anti-CD11b antibodies of the disclosure includes 1, 2, or 3 light chain CDR sequences that are or include a sequence of SEQ ID NOs: 8, 9, and/or 10. In some embodiments, the humanized forms of mAb107 or an antigen-binding antibody fragment thereof includes one or more sequences including SEQ ID NOs: 1-18. In some embodiments, the humanized forms of mAb107 or an antigen-binding antibody fragment includes a sequence that is at least 95%, 96%, 97%, 98%, 99%, 99.1%, 99.2%, 99.3%, 99.4%, 99.5%, 99.6%, 99.7%, 99.8%, or 99.9% identical to SEQ ID NOs: 1-18.

Amino acid sequences of a humanized form of mAb107 antibody or antigen-binding fragment binds of the present disclosure may be substituted through conservative substitution. The term “conservative substitution” used herein refers to modification of a polypeptide in which one or more amino acids are substituted with an amino acid having a similar biochemical property so as not to cause the loss of a biological or biochemical function of the corresponding polypeptide. The term “conservative sequence variant” or “conservative amino acid substitution” used herein is the substitution of an amino acid residue with an amino acid residue having a similar side chain. Amino acid residues having a similar side chain are defined in the art. Those residues encompass amino acids with a basic side chain (e.g., lysine, arginine, and histidine), amino acids with an acidic side chain (e.g., aspartic acid and glutamate), amino acids with a non-charged polar side chain (e.g., glycine, asparagine, glutamine, serine, threonine, tyrosine, and cysteine), amino acids with a non-polar side chain (e.g., alanine, valine, leucine, isoleucine, proline, phenylalanine, methionine, and tryptophan), amino acids with a betabranched side chain (e.g., threonine, valine, and isoleucine) and amino acids with an aromatic side chain (e.g., tyrosine, phenylalanine, tryptophan, and histidine). Therefore, it is expected that the antibody of the present invention can have conservative amino acid substitution, and still ensure an activity.

In some embodiments, the disclosure provides polynucleotides comprising a nucleotide sequence encoding humanized forms of mAb107 and fragments thereof. Humanized forms of mAb107 antibodies and fragments therefore may be produced from nucleic acid molecules using molecular biological methods known to the art. Nucleic acids of the present disclosure include, for example DNA and/or RNA.

Nucleic acid constructs of the present disclosure may be inserted into an expression vector or viral vector by methods known to the art, and nucleic acid molecules may be operably linked to an expression control sequence. A vector comprising any of the above described nucleic acid molecules, or fragments thereof, is further provided by the present disclosure. Any of the above nucleic acid molecules, or fragments thereof, can be cloned into any suitable vector and can be used to transform or transfect any suitable host. The selection of vectors and methods to construct them are commonly known to persons of ordinary skill in the art and are described in general technical references (see, in general, “Recombinant DNA Part D,” Methods in Enzymology, Vol. 153, Wu and Grossman, eds., Academic Press (1987)).

In some embodiments, conventionally used techniques, such as, for example, electrophoresis, calcium phosphate precipitation, DEAE-dextran transfection, lipofection, etc. may be used to introduce a foreign nucleic acid (DNA or RNA) into a prokaryotic or eukaryotic host cell. Desirably, a vector may include regulatory sequences, such as transcription and translation initiation and termination codons, which are specific to the type of host (e.g., bacterium, fungus, plant or animal) into which the vector is to be introduced, as appropriate and taking into consideration whether the vector is DNA or RNA In some embodiments, a vector comprises regulatory sequences that are specific to the genus of the host. Preferably, a vector comprises regulatory sequences that are specific to the species of the host.

Suitable viral vectors include, for example, retroviral vectors, parvovirus-based vectors, e.g., adeno-associated virus (AAV)-based vectors, AA V-adenoviral chimeric vectors, and adenovirus-based vectors, and lentiviral vectors, such as Herpes simplex (HSV)-based vectors. These viral vectors can be prepared using standard recombinant DNA techniques described in, for example, Sambrook et al., Molecular Cloning, a Laboratory Manual, 2d edition, Cold Spring Harbor Press, Cold Spring Harbor, N. Y (1989); and Ausubel et al., Current Protocols in Molecular Biology, Greene Publishing Associates and John Wiley & Sons, New York, N.Y (1994).

In some embodiments, nucleic acid molecules are inserted into a vector that is able to express humanized mAb107 antibody or fragment thereof when introduced into an appropriate host cell. Appropriate host cells include, but are not limited to, bacterial, yeast, insect, and mammalian cells. Exemplary host cells include prokaryotes (e.g., E. col) and eukaryotes (e.g., a COS or a CHO cell). Mammalian host cells that could be used include human Hela 293, Expi293, H9 and Jurkat cells, mouse NIH3T3 and C 127 cells, Cos 1, Cos 7 and CV 1, quail QCl-3 cells, mouse L cells and Chinese hamster ovary (CHO) cells (e.g., DG44 cells). In some embodiments, a mammalian host cell suitable for the expression of the antibody may be a Chinese Hamster Ovary (CHO) cell (for example, including DHFR-CHO cells used along with a DHFR-selectable marker), an NSO myeloma cell, a COS cell or an SP2 cell.

In some embodiments, nucleic acids and vectors of the present disclosure may be isolated and/or purified. The present disclosure also provides a composition comprising an above-described isolated or purified nucleic acid molecule, optionally in the form of a vector. Isolated nucleic acids and vectors may be prepared using standard techniques known in the art including, for example, alkali/SDS treatment, CsCl binding, column chromatography, agarose gel electrophoresis and other techniques well known in the art. The composition can comprise other components as described further herein.

The mouse IgG1 mAb107 (mAb107) blocks multiligand binding to CD11b, and CD11b-mediated transendothelial neutrophil/monocyte migration and phagocytosis (Li et al., J Immunol. 2002 Feb. 1; 168(3):1219-25). Crystal structure determination of mouse mAb107 in complex with CD11b revealed that the mAb is a ligand-mimic, occupying the ligand-binding site in CD11b. An unexpected feature of the structure, conformed in the cellular integrin, was that binding of mAb107 stabilized the integrin in its inactive non-signaling conformation, a first example for an integrin inhibitor (Mahalingam et al., J Immunol. 2011 Dec. 15; 187(12):6393-401). Most recently, mAb107 has been shown to protect native kidneys from otherwise irreversible kidney failure caused by severe ischemia-reperfusion injury (IRI) in non-human primates (NHP) (Dehnadi et al., Nat Commun. 2017 Jan. 10; 8:13899). mAb107 blocked leukocyte homing the reperfused organ, phagocytosis and production of IL-18, RANTES, C3, IL-6 and IFN-γ proteins in the ischemic-reperfused NHP kidneys.

IRI is common acute inflammatory response mediated by the activated innate immune cells that infiltrate the ischemic organ following reperfusion. It underlies post-ischemic failure of multiple organs including the heart, brain and kidney. It is also the cause of delayed graft function and primary allograft non-function, which have been widely acknowledged to have detrimental effects on graft survival, initial length of stay, medical costs, and mortality (Siedlecki et al., Kidney Int. 2011 August; 80(3):263-71; Menke et al., Curr Opin Organ Transplant. 2014 August; 19(4):395-400; Cooper et al., Curr Opin Nephrol Hypertens. 2013 November; 22(6):698-703; Renders and Heeman, Curr Opin Organ Transplant. 2012 December; 17(6):634-9). Previous efforts to limit IRI, conducted primarily in rodents, have targeted individual proinflammatory mediators and showed promise in rodents, but this approach was less effective in large animals and failed in human trials (Cerda et al., Clin J Am Soc Nephrol. 2015 Oct. 7; 10(10):1859-67; Molitoris et al., Clin. J. Am. Soc. Nephrol. 7, 842-843 (2012); Anderson et al., Cold Spring Harb Perspect Med. 2013 Sep. 1; 3(9):a015503; Gallagher et al., Expert Opin Investig Drugs. 2017 February; 26(2):141-154; Benoit et al., Pediatr Nephrol. 2018 May; 33(5):779-787). Contributory factors likely included the significant species differences in immune responses between rodents and primates (Seok et al., Proc Natl Acad Sci USA. 2013 Feb. 26; 110(9):3507-12), the heterogeneity of immune cell populations in primates, and the dual roles of innate immune cells not only in tissue injury but also in tissue repair, which complicates target selection and timing of interventions. Thus the impressive success of mAb107 in preventing post-ischemic kidney failure in nonhuman primates is a major advance, forming a convincing rationale for testing its utility in humans. Described herein are antibodies or antigen-binding fragments thereof, comprising humanized and mutagenized antibodies or antigen-binding fragments thereof that bind CD11b.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antibody” encompasses not only intact polyclonal or monoclonal antibodies, but also any antigen binding fragment (i.e., “antigen-binding portion”) or single chain thereof, fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known. Exemplary antibodies and antibody fragments include, but are not limited to, monoclonal antibodies (including full-length monoclonal antibodies), polyclonal antibodies, multispecific antibodies formed from at least two different epitope binding fragments (e.g., bispecific antibodies), camelid antibodies, chimeric antibodies, single chain variable fragments (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the invention), intrabodies, and epitope-binding fragments of any of the above.

An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains of antibody, wherein these domains are present in a single polypeptide chain. Generally the Fv polypeptide further comprises a polypeptide linker between the VH and VL domains, which enables the scFv to form the desired structure for antigen binding. One exemplary scFV is shown in SEQ ID NO:15. This scFV includes a humanized VH segment, a humanized VL segment, and a linker between these two segments.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific. Antibodies and antibody fragments of the present disclosure can be modified in the Fc region to provide desired effector functions or serum half-life.

In some embodiments, the Fc region can be conjugated to PEG or albumin to increase the serum half-life, or some other conjugation that results in the desired effect. Alternatively, where it is desirable to eliminate or reduce effector function, so as to minimize side effects or therapeutic complications, certain other Fc regions may be used

### Methods of Use

The disclosure provides methods of treatment that include administering to a subject a composition disclosed herein.

The term “subject,” as used herein, refers to any animal. In some embodiments, the subject is a mammal. In some embodiments, the term “subject”, as used herein, refers to a human (e.g., a man, a woman, or a child). Samples for use in the methods can include serum samples, e.g., obtained from the selected subject.

In some embodiments, subject selection can include obtaining a sample from a subject (e.g., a candidate subject) and testing the sample for an indication that the subject is suitable for selection. In some embodiments, the subject can be confirmed or identified, e.g. by a health care professional, as having had or having a condition or disease. In some embodiments, exhibition of a positive response towards a condition or disease can be made from patient records, family history, and/or detecting an indication of a positive response. In some embodiments multiple parties can be included in subject selection. For example, a first party can obtain a sample from a candidate subject and a second party can test the sample. In some embodiments, subjects can be selected and/or referred by a medical practitioner (e.g., a general practitioner). In some embodiments, subject selection can include obtaining a sample from a selected subject and storing the sample and/or using the in the methods disclosed herein. Samples can include, for example, cells or populations of cells.

The antibodies described herein can be administered to patients that have suffered an acute myocardial infarction in the hopes of preventing damage due to ischemia reperfusion injury. In some embodiments, the myocardial infarction condition is post myocardial infarction left ventricular adverse remodeling. In some embodiments, the myocardial infarction condition includes myocardial stunning. In some embodiments, the myocardial infarction condition includes microvascular dysfunction. In some embodiments, the disclosed anti-CD11b antibodies are administered to a subject with an ischemia reperfusion injury within about 5 hours of the injury. In some embodiments, the disclosed anti-CD11b antibodies are administered to a subject with an ischemia reperfusion injury within about 2 hours of the injury. In some embodiments, the disclose anti-CD11b antibodies are administered prior to the ischemic reperfusion injury. Currently it is estimated that more than 30% of the 860,000 myocardial infarction patients in the U.S. each year suffer complications due to reperfusion injury and it is a chief cause of adverse outcomes.

The antibodies described herein can also be used in preventing acute kidney injury (AKI) caused by ischemia reperfusion injury that complicates recovery from cardiac surgery in up to 30-40% of patients, totaling an estimated 1.8 million patients in the U.S each year and at an annual cost to the U.S. health system of approximately $10 billion. Renal replacement therapy (dialysis) occurs in 2-5% of such patients and is associated with 50% mortality, and with increased risk of long-term mortality (HR, 1.31; 95% CI, 1.16-1.47; p<0.00001), with no effective preventive therapy. This clinical setting resembles and is supported by results in the nonhuman primate model of renal ischemia/reperfusion injury (Dehnadi et al., Nat Commun. 2017 Jan. 10; 8:13899) in being elective, common, causing a relatively standardized insult and where close monitoring is available. In some embodiments, the kidney injury includes kidney fibroinflammatory disease. In some embodiments, the kidney injury includes post-ischemic renal fibrosis. In some embodiments, the disclosed anti-CD11b antibodies are administered to a subject with an ischemia reperfusion injury within about 5 hours of the injury. In some embodiments, the disclosed anti-CD11b antibodies are administered to a subject with an ischemia reperfusion injury within about 2 hours of the injury. In some embodiments, the disclose anti-CD11b antibodies are administered prior to the ischemic reperfusion injury.

The antibodies described herein can also be used to prevent (reduce risk of) delayed graft function (DGF, orphan drug designation approved by FDA) resulting from ischemia reperfusion injury of the kidney graft. This is a common and major complication in kidney transplants, rising in 2015 to 31% of patients receiving allografts from deceased donors. In 2016, of the 19,060 kidney transplants performed in the US alone, 70.5% (˜13,437 per year) came from deceased donors, and ˜14,000 per year in the EU. There is no approved therapy. Again, the clinical situation here resembles the experimental set up in a nonhuman primate model (Dehnadi et al., Nat Commun. 2017 Jan. 10; 8:13899). DGF increases post-surgical costs by more than $4,000, increases posttransplant hospital stay (usually 5-10 days) by up to 75%, and over five years post-transplant, DGF patients had significantly higher time on dialysis, transplant rejection, and mortality. mAb107 for DGF could reach $300M+ annual revenues, with less than one-third transplant penetration for hospital treatment, and one-third of those moving on to home treatment.

The antibodies of the disclosure can also be used to improve the acceptance of xenografts and in tolerance induction to allografts when delivered in combination with anti-CD40 antibodies. Treatment with anti-CD11b antibodies mitigates early xenograft rejection in animal models, and anti-CD40/CD11 b therapy is statistically similar to anti-CD154 antibody treatment for preventing xenotransplantation rejection (Liu and Ford, Am J Transplant. 2020; 20:2216-2225; D. A. Faber et al., “Combined CD11b/CD40 Blockade is Superior to CD40 Blockade Alone in Prolonging Survival in Pig-to-Nonhuman Primate Renal Xenotransplantation,” Abstract 2 at 2021 American Transplant Congress).

The antibodies of the disclosure can also be administered to a subject with a reperfusion injury of the lungs. For example, the antibodies of the disclosure can be administered to a subject having pulmonary fibrosis. In some embodiments, the disclosed anti-CD11b antibodies are administered to a subject with a reperfusion injury within about 5 hours of the injury. In some embodiments, the disclosed anti-CD11b antibodies are administered to a subject with a reperfusion injury within about 2 hours of the injury. In some embodiments, the disclose anti-CD11b antibodies are administered prior to the reperfusion injury.

The antibodies of the disclosure can also be administered to subjects with an autoimmune disease, for example cytoplasmic antineutrophil cytoplasmic antibodies (cANCA)-associated vasculitis or diabetic nephropathy. Additionally the antibodies of the disclosure can also be administered to subjects undergoing or having undergone chemotherapy.

The antibodies of the disclosure can also be administered to a subject having or diagnosed with dry eye disease. In some embodiments, the antibodies of the disclosure can be administered topically. In some embodiments, the antibodies of the disclosure can be administered to the cornea. In some embodiments, the antibodies of the disclosure can be administered to the conjunctival epithelium. Topical application of risuteganib, which blocks leukocyte homing in part through CD11b, reduces inflammation and improves the signs and symptoms of dry eye disease. (Donnenfeld, et al., “Prospective, Randomized, Double-Masked, Vehicle-Controlled, Safety and Efficacy Study of Topical Risuteganib in Treating Dry Eye Disease,” ASCRS 2021 meeting, paper 77664).

The antibodies of the disclosure can also be administered to a subject having or diagnosed with Alzheimer's disease. In some embodiments, the antibodies of the disclosure can be administered with or without other components to decrease microglia activation. Microglia activation mediates early neuronal synapse loss in Alzheimer mouse models. (Hong et al., Science. 2016 May 6. 352:6286; Merlini et al., Neuron. 2019 Mar. 20. 101:1099-1108). Therefore, blocking or attenuating microglia activation through administration of the anti-CD11b antibodies of the disclosure can be used to treat a subject having or diagnosed with Alzheimer's disease.

The antibodies described herein include humanized versions of mAb107, that can include one or more amino-acid substitution, based on our crystal structure, include those that improve affinity of humanized mAb107 to CD11b vs. the mouse version.

See also, US 2018/0244782, U.S. Pat. Nos. 7,323,552, 7,998,738, and 10,738,121, the entire disclosures of which are incorporated herein by reference.

### Pharmaceutical Compositions and Methods of Administration

The methods described herein include the use of pharmaceutical compositions comprising the antibodies or antigen-binding fragments thereof described herein as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Examples

The invention is further described in the following examples, which do not limit the scope of the invention described in the claims.

### Example 1. Humanizing mAb107

Brief Methods

Design: The sequences flanking the three CDRs in each variable and heavy and light chain of the mouse mAb107 were changed into human immunoglobulin sequences. In addition, we introduced a change in CDR2 of the variable heavy chain replacing asparagine (N) with an aspartic acid (D) to improve affinity. cDNAs were made commercially.

Cloning: Variable regions of our designed cDNAs encoding the variable heavy and light chain of humanized mAb107 were reverse translated using online program, and synthesized by IDT. Secretion signal sequence was added at 5′-end of each chain. The N/D mutation was introduced to the heavy chain using QuikChange II XL site-directed mutagenesis kit (Agilent). The variable region of the heavy chain was joined to human IgG4K constant region by overlapping PCR, and cloned into pcDNA3.4 using XbaI and XhoI restriction sites. Similarly, light chain variable region was fused to the human kappa light constant region.

Production, purification, and evaluation: Expi293 cells (Thermo Fisher) were used to produce recombinant antibodies. Cells were grown on orbital shaker (125 rpm) in Expi293 Expression Medium (Thermo Fisher) at 37° C. in 5% CO2. Expi293 cells were transiently transfected with 1:1 heavy chain and light chain plasmids, using manufacturer's protocol. Culture supernatant was harvested after one week, and IgG was purified using Protein G agarose beads (Thermo Scientific). We routinely obtained 1-3 mg of purified IgG per 100 ml of cell culture. The protein sizes of the heavy and light chains and purity of the IgG were confirmed on SDSPAGE. Affinity of the humanized mAb107 vs. mouse mAb107 was measured using the A-domain of CD11b as ligand by Bio-layer interferometry (BLI) using the Octet Assay system.

Results

The optimized amino acid sequences of heavy and light chains of humanized N55D-substituted mAb107 are as follows:

A) Humanized Heavy Chain (CDR1, 2 and 3 are in bold;
N57D substitution in CDR2 indicated in lower case).
(SEQ ID NO: 1)
QVQLVQSGAEVKKPGASVKVSCKPSGFNIKDIYMQWVRQAPGQRLEWIGRIDPAdDKTKY 60
DPKFQGRATITADTSASTAYLELSSLRSEDTAVYYCASEGHYGYDGYAMDYWGQGTTVTV 120
SS 122
B) Humanized Light Chain (CDR1, 2 and 3 are in bold)
(SEQ ID NO: 2)
DIVMTQSPDSLAVSLGERATINCKSSQNLLYSSNQKNYLAWYQQKPGOPPKLLIYWASTR 60
ESGVPDRFSGSGSGTDFTLTISSLQAEDVAVYYCQQYYSYPLTFGQGTKLEIK 113

Sequences of Humanized mAb107 IgG4κ

Heavy (H) chain (CDRs in bold; N57D substitution in CDR2 indicated in lower case; constant region in italics). The lower case “d” in CDR3 is the D residue that binds the metal ion at MIDAS.

(SEQ ID NO: 3)
QVQLVQSGAEVKKPGASVKVSCKPSGFNIKDIYMQWVRQAPGQRLEWIGR
IDPAdDKTKYDPKFQGRATITADTSASTAYLELSSLRSEDTAVYYCASEG
HYGYdGYAMDYWGQGTTVTVSSASTKGPSVFPLAPCSRSTSESTAALGCL
VKDYFPEPVTVSWNSGALTSGVHTFPAVLQSSGLYSLSSVVTPSSSLGTK
TYTCNVDHKPSNTKVDKR
mAB107 IgG1 is
(SEQ ID NO: 17)
QVQLVQSGAEVKKPGASVKVSCKPSGFNIKDIYMQWVRQAPGQRLEWIGR
IDPAdDKTKYDPKFQGRATITADTSASTAYLELSSLRSEDTAVYYCASEG
HYGYdGYAMDYWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSC
Light (L) chain (CDRs in bold; constant region in
italics)
(SEQ ID NO: 4)
DIVMTQSPDSLAVSLGERATINCKSSQNLLYSSNQKNYLAWYQQKPGQPP
KLLIYWASTRESGVPDRFSGSGSGTDFTLTISSLQAEDVAVYYCQQYYSY
PLTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSENRGEC

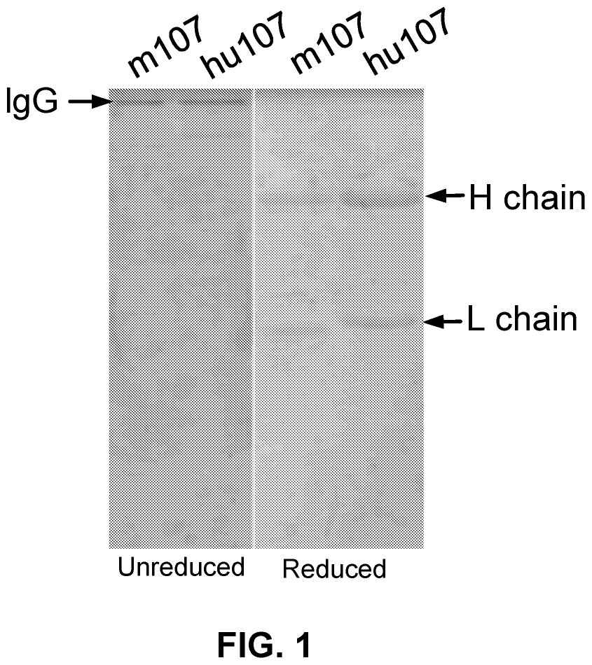

The protein sizes of the heavy and light chains and purity of the IgG were confirmed on SDSPAGE, and the expected sizes were seen (FIG. 1).

Affinity for CD11b A-domain was determined by BLI. The affinity for the parental antibody, Mouse mAb107, was 1-5 nM. The affinity of the humanized humAb107 was 5-9 nM.

### Example 2. Humanized Mutagenized Anti-CD11b

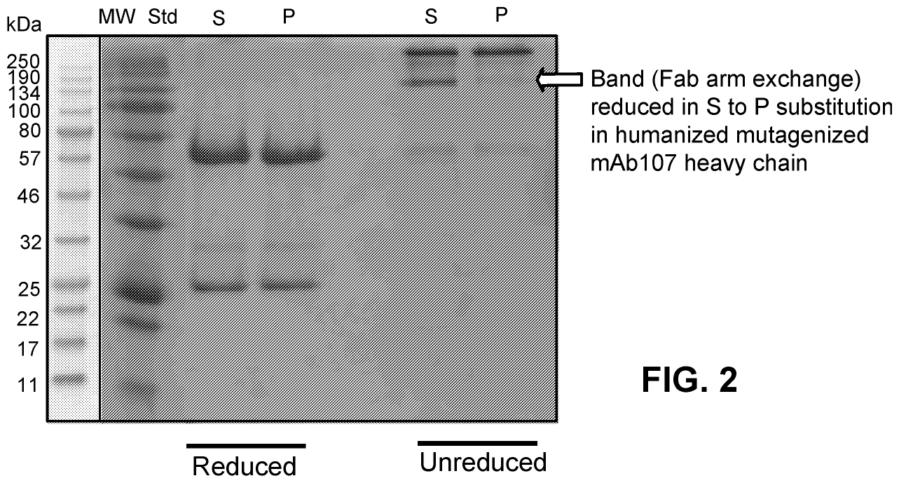

An S to P mutation at position [228] in the IgG4 hinge region (VESKYGPPCPPCPAPEFLGG)(italicized in (SEQ ID NO: 12) reduced Fab arm exchange, as shown in FIG. 2.

```
DNA sequence of Humanized mutagenized mAb107 IgG4k heavy chain V5 -
affinity 5-9 nM
(SEQ ID NO: 11)
atggagacagacacactcctgctatgggtactgctgctctgggttccaggttccactggtgaccaggtgca
gctggtgcagagcggcgcggaagtgaagaaaccgggcgcgagcgtgaaagtgagctgcaagccgagcggct
ttaacattaaagatatttatatgcagtgggtgaggcaggcgccgggccagcgcctggaatggattggccgc
attgatccggcggacgataaaaccaaatatgatccgaaatttcagggccgcgcgaccattaccgcggatac
cagcgcgagcaccgcgtatctggaactgagcagcctgcgcagcgaagataccgcggtgtattattgcgcga
gcgaaggccattatggctatgatggctatgcgatggattattggggccagggcaccaccgtgaccgtgagc
agcgctagcaccaagggcccatccgtcttccccctggcgccctgctccaggagcacctccgagagcacagc
cgccctgggctgcctggtcaaggactacttccccgaaccggtgacggtgtcgtggaactcaggcgccctga
ccagcggcgtgcacaccttcccggctgtcctacagtcctcaggactctactccctcagcagcgtggtgacc
gtgccctccagcagcttgggcacgaagacctacacctgcaacgtagatcacaagcccagcaacaccaaggt
ggacaagagagttgagtccaaatatggtcccccatgcccaccatgcccagcacctgagttcctggggggac
catcagtcttcctgttccccccaaaacccaaggacactctcatgatctcccggacccctgaggtcacgtgc
gtggtggtggacgtgagccaggaagaccccgaggtccagttcaactggtacgtggatggcgtggaggtgca
taatgccaagacaaagccgcgggaggagcagttcaacagcacgtaccgtgtggtcagcgtcctcaccgtcc
tgcaccaggactggctgaacggcaaggagtacaagtgcaaggtctccaacaaaggcctcccgtcctccatc
gagaaaaccatctccaaagccaaagggcagccccgagagccacaggtgtacaccctgcccccatcccagga
ggagatgaccaagaaccaggtcagcctgacctgcctggtcaaaggcttctaccccagcgacatcgccgtgg
agtgggagagcaatgggcagccggagaacaactacaagaccacgcctcccgtgctggactccgacggctcc
ttcttcctctacagcaggctaaccgtggacaagagcaggtggcaggaggggaatgtcttctcatgctccgt
gatgcatgaggctctgcacaaccactacacacagaagagcctctccctgtctctgggtaaatga
Protein Translation:
atg gag aca gac aca ctc ctg cta tgg gta ctg ctg ctc tgg gtt cca ggt tcc act ggt
  M E T D T L L L W V L L L W V P G S T G
gac cag gtg cag ctg gtg cag agc ggc gcg gaa gtg aag aaa ccg ggc gcg agc gtg aaa
  D Q V Q L V Q S G A E V K K P G A S V K
gtg agc tgc aag ccg agc ggc ttt aac att aaa gat att tat atg cag tgg gtg agg cag
  V S C K P S G F N I K D I Y M Q W V R Q
gcg ccg ggc cag cgc ctg gaa tgg att ggc cgc att gat ccg gcg gac gat aaa acc aaa
  A P G Q R L E W I G R I D P A D D K T K
tat gat ccg aaa ttt cag ggc cgc gcg acc att acc gcg gat acc agc gcg agc acc gcg
  Y D P K F Q G R A T I T A D T S A S T A
tat ctg gaa ctg agc agc ctg cgc agc gaa gat acc gcg gtg tat tat tgc gcg agc gaa
  Y L E L S S L R S E D T A V Y Y C A S E
ggc cat tat ggc tat gat ggc tat gcg atg gat tat tgg ggc cag ggc acc acc gtg acc
  G H Y G Y D G Y A M D Y W G Q G T T V T
gtg agc agc gct agc acc aag ggc cca tcc gtc ttc ccc ctg gcg ccc tgc tcc agg agc
  V S S A S T K G P S V F P L A P C S R S
acc tcc gag agc aca gcc gcc ctg ggc tgc ctg gtc aag gac tac ttc ccc gaa ccg gtg
  T S E S T A A L G C L V K D Y F P E P V
acg gtg tcg tgg aac tca ggc gcc ctg acc agc ggc gtg cac acc ttc ccg gct gtc cta
  T V S W N S G A L T S G V H T F P A V L
cag tcc tca gga ctc tac tcc ctc agc agc gtg gtg acc gtg ccc tcc agc agc ttg ggc
  Q S S G L Y S L S S V V T V P S S S L G
acg aag acc tac acc tgc aac gta gat cac aag ccc agc aac acc aag gtg gac aag aga
  T K T Y T C N V D H K P S N T K V D K R
gtt gag tcc aaa tat ggt ccc cca tgc cca cca tgc cca gca cct gag ttc ctg ggg gga
  V E S K Y G P P C P P C P A P E F L G G
cca tca gtc ttc ctg ttc ccc cca aaa ccc aag gac act ctc atg atc tcc cgg acc cct
  P S V F L F P P K P K D T L M I S R T P
gag gtc acg tgc gtg gtg gtg gac gtg agc cag gaa gac ccc gag gtc cag ttc aac tgg
  E V T C V V V D V S Q E D P E V Q F N W
tac gtg gat ggc gtg gag gtg cat aat gcc aag aca aag ccg cgg gag gag cag ttc aac
  Y V D G V E V H N A K T K P R E E Q F N
agc acg tac cgt gtg gtc agc gtc ctc acc gtc ctg cac cag gac tgg ctg aac ggc aag
  S T Y R V V S V L T V L H Q D W L N G K
gag tac aag tgc aag gtc tcc aac aaa ggc ctc ccg tcc tcc atc gag aaa acc atc tcc
  E Y K C K V S N K G L P S S I E K T I S
aaa gcc aaa ggg cag ccc cga gag cca cag gtg tac acc ctg ccc cca tcc cag gag gag
  K A K G Q P R E P Q V Y T L P P S Q E E
atg acc aag aac cag gtc agc ctg acc tgc ctg gtc aaa ggc ttc tac ccc agc gac atc
  M T K N Q V S L T C L V K G F Y P S D I
gcc gtg gag tgg gag agc aat ggg cag ccg gag aac aac tac aag acc acg cct ccc gtg
  A V E W E S N G Q P E N N Y K T T P P V
ctg gac tcc gac ggc tcc ttc ttc ctc tac agc agg cta acc gtg gac aag agc agg tgg
  L D S D G S F F L Y S R L T V D K S R W
cag gag ggg aat gtc ttc tca tgc tcc gtg atg cat gag gct ctg cac aac cac tac aca
  Q E G N V F S C S V M H E A L H N H Y T
cag aag agc ctc tcc ctg tct ctg ggt aaa tga (SEQ ID NO: 11)
  Q K S L S L S L G K - (SEQ ID NO: 12)
Leader sequence:
(SEQ ID NO: 13)
METDTLLLWVLLLWVPGSTGD
N to D substitution in CDR2H: in bold (to enhance affinity) [N57 in the
crystal structure of 107Fab/CD11bA domain complex].
S to P substitution: in bold (to prevent Fab arm exchange [JBC, VOL. 290,
NO. 9, pp. 5462-5469, Feb. 27, 2015]).
DNA sequence of Humanized mAb107 IgG4k light chain
(SEQ ID NO: 14)
Atggagacagacacactcctgctatgggtactgctgctctgggttccaggttccactggtgacgatattgt
gatgacccagagcccggatagcctggcggtgagcctgggcgaacgcgcgaccattaactgcaaaagcagcc
agaacctgctgtatagcagcaaccagaaaaactatctggcgtggtatcagcagaaaccgggccagccgccg
aaactgctgatttattgggcgagcacccgcgaaagcggcgtgccggatcgctttagcggcagcggcagcgg
caccgattttaccctgaccattagcagcctgcaggcggaagatgtggcggtgtattattgccagcagtatt
atagctatccgctgacctttggccagggcaccaaactggaaattaaacgtacggtggctgcaccatctgtc
ttcatcttcccgccatctgatgagcagttgaaatctggaactgcctctgttgtgtgcctgctgaataactt
ctatcccagagaggccaaagtacagtggaaggtggataacgccctccaatcgggtaactcccaggagagtg
tcacagagcaggacagcaaggacagcacctacagcctcagcagcaccctgacgctgagcaaagcagactac
gagaaacacaaagtctacgcctgcgaagtcacccatcagggcctgagctcacccgtcacaaagagcttcaa
caggggagagtgttag
Protein Translation:
atg gag aca gac aca ctc ctg cta tgg gta ctg ctg ctc tgg gtt cca ggt tcc act ggt
  M E T D T L L L W V L L L W V P G S T G
gac gat att gtg atg acc cag agc ccg gat agc ctg gcg gtg agc ctg ggc gaa cgc gcg
  D D I V M T Q S P D S L A V S L G E R A
acc att aac tgc aaa agc agc cag aac ctg ctg tat agc agc aac cag aaa aac tat ctg
  T I N C K S S Q N L L Y S S N Q K N Y L
gcg tgg tat cag cag aaa ccg ggc cag ccg ccg aaa ctg ctg att tat tgg gcg agc acc
  A W Y Q Q K P G Q P P K L L I Y W A S T
cgc gaa agc ggc gtg ccg gat cgc ttt agc ggc agc ggc agc ggc acc gat ttt acc ctg
  R E S G V P D R F S G S G S G T D F T L
acc att agc agc ctg cag gcg gaa gat gtg gcg gtg tat tat tgc cag cag tat tat agc
  T I S S L Q A E D V A V Y Y C Q Q Y Y S
tat ccg ctg acc ttt ggc cag ggc acc aaa ctg gaa att aaa cgt acg gtg gct gca cca
  Y P L T F G Q G T K L E I K R T V A A P
tct gtc ttc atc ttc ccg cca tct gat gag cag ttg aaa tct gga act gcc tct gtt gtg
  S V F I F P P S D E Q L K S G T A S V V
tgc ctg ctg aat aac ttc tat ccc aga gag gcc aaa gta cag tgg aag gtg gat aac gcc
  C L L N N F Y P R E A K V Q W K V D N A
ctc caa tcg ggt aac tcc cag gag agt gtc aca gag cag gac agc aag gac agc acc tac
  L Q S G N S Q E S V T E Q D S K D S T Y
agc ctc agc agc acc ctg acg ctg agc aaa gca gac tac gag aaa cac aaa gtc tac gcc
  S L S S T L T L S K A D Y E K H K V Y A
tgc gaa gtc acc cat cag ggc ctg agc tca ccc gtc aca aag agc ttc aac agg gga gag
  C E V T H Q G L S S P V T K S F N R G E
tgt tag (SEQ ID NO: 14)
  C - (SEQ ID NO: 18)
MW = 24,569
Leader Sequence:
(SEQ ID NO: 13)
METDTLLLWVLLLWVPGSTGD
scFv version of humanized mutated mAB107
(SEQ ID NO: 15)
EFQVQLVQSGAEVKKPGASVKVSCKPSGFNIKDIYMOWVROAPGORLEWIGRIDPADDKTKYDPKFQGRATITADTSAS
TAYLELSSLRSEDTAVYYCASEGHYGYDGYAMDYWGQGTTVTVSSGGGGSGGGGSGGGGSDIVMTQSPDSLAVSLGERA
TINCKSSQNLLYSSNQKNYLAWYQQKPGQPPKLLIYWASTRESGVPDRFSGSGSGTDFTLTISSLQAEDVAVYYCQQYY
SYPLTFGQGTKLEIKENLYFQGS
Features:
Two foreign amino acids: EF
GS Linker:
(SEQ ID NO:16)
GGGGSGGGGSGGGGS
CDR1-3 of Heavy and light chain are in bold
```

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof, comprising an amino acid sequence that comprises the following complementarity determining regions (CDR):
1) CDR 1 of the VH of SEQ ID NO:1, comprising a sequence of SEQ ID NO: 5;
2) CDR 1 of the VL of SEQ ID NO:2, comprising a sequence of SEQ ID NO: 8;
3) CDR 2 of the VH of SEQ ID NO:1, comprising a sequence of SEQ ID NO: 6;
4) CDR 2 of the VL of SEQ ID NO:2, comprising a sequence of SEQ ID NO: 9;
5) CDR3 of the VH of SEQ ID NO:1, comprising a sequence of SEQ ID NO: 7; and
6) CDR 3 of the VL of SEQ ID NO:2, comprising a sequence of SEQ ID NO: 10.

2. An antibody or antigen-binding fragment thereof, comprising a heavy chain variable region comprising VH CDRs 1, 2, 3, and a light chain variable region comprising VL CDRs 1, 2, 3, wherein
the VH CDRs 1, 2, 3 are identical to complementarity determining regions in SEQ ID NO: 1, and
the VL CDRs 1, 2, 3 are identical to complementary determining regions in SEQ ID NO: 2.

3. An antibody or antigen-binding fragment thereof 2, which comprises an amino acid sequence that comprises an amino acid sequence at least 95% identical to an amino acid sequence selected from the group consisting of:
(SEQ ID NO: 3)
QVQLVQSGAEVKKPGASVKVSCKPSGFNIKDIYMQWVRQAPGQRLEWIGR
IDPAdDKTKYDPKFQGRATITADTSASTAYLELSSLRSEDTAVYYCASEG
HYGYdGYAMDYWGQGTTVTVSSASTKGPSVFPLAPCSRSTSESTAALGCL
VKDYFPEPVTVSWNSGALTSGVHTFPAVLQSSGLYSLSSVVTPSSSLGTK
TYTCNVDHKPSNTKVDKR
(heavy chain, SEQ ID NO: 17)
QVQLVQSGAEVKKPGASVKVSCKPSGFNIKDIYMQWVRQAPGQRLEWIGR
IDPAdDKTKYDPKFQGRATITADTSASTAYLELSSLRSEDTAVYYCASEG
HYGYDGYAMDYWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSC
(heavy chain, SEQ ID NO: 13)
QVQLVQSGAEVKKPGASVKVSCKPSGFNIKDIYMQWVRQAPGQRLEWIGR
IDPADDKTKYDPKFQGRATITADTSASTAYLELSSLRSEDTAVYYCASEG
HYGYDGYAMDYWGQGTTVTVSSASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK,
and
(light chain, SEQ ID NO: 4)
DIVMTQSPDSLAVSLGERATINCKSSQNLLYSSNQKNYLAWYQQKPGQPP
KLLIYWASTRESGVPDRFSGSGSGTDFTLTISSLQAEDVAVYYCQQYYSY
PLTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC,
provided that the complementarity-determining regions of the sequence are not altered.

4. The antibody or antigen-binding fragment thereof of claim 1, wherein the antigen-binding fragment is a single chain variable fragment (scFv).

5. The antibody or antigen-binding fragment thereof of claim 6, comprising SEQ ID NO:15.

6. A method of ameliorating a pathology associated with ischemia reperfusion injury in a subject comprising administering to the subject a composition comprising a therapeutically effective amount of an antibody or antigen-binding fragment thereof of claims 1-5.

7. The method of claim 6, wherein the pathology is post-ischemic renal fibrosis.

8. The method of claim 6, wherein the pathology is a kidney fibroinflammatory disease.

9. The method of claim 6, wherein the pathology is pulmonary fibrosis.

10. The method of claim 6, wherein the pathology is post-myocardial infarction left ventricular adverse remodeling.

11. The method of claim 6, wherein the composition is administered to the subject within about 5 hours after the ischemia reperfusion injury.

12. The method of claim 6, wherein the composition is administered to the subject within about 2 hours after the ischemia reperfusion injury.

13. A method of treating a subject having or at risk of developing a disorder associated with ischemia reperfusion injury in an organ comprising:
administering to the subject a composition comprising a therapeutically effective amount of an antibody or antigen-binding fragment thereof of claims 1-5.

14. The method of claim 13, wherein the organ is a kidney.

15. The method of claim 14, wherein the disorder is acute kidney injury.

16. The method of claim 13, wherein the organ is a heart.

17. The method of claim 16, wherein the disorder is acute coronary syndrome.

18. The method of claim 16, wherein the disorder is acute myocardial infarction (NI).

19. The method of claim 13, wherein the organ is a lung.

20. The method of claim 13, wherein the composition is administered to the subject within about 5 hours after the ischemia reperfusion injury.

21. The method of claim 13, wherein the composition is administered to the subject within about 2 hours after the ischemia reperfusion injury.

22. A method of providing an organ for transplantation, comprising:
administering to an organ donor a composition comprising an antibody or antigen-binding fragment thereof of claims 1-5; and
harvesting the organ from the organ donor.

23. The method of claim 22, wherein the organ is a kidney, a heart, or a lung.

24. A method of reducing delayed graft function following organ transplantation, comprising
administering to an organ recipient a therapeutically effective amount of composition comprising an antibody or antigen-binding fragment thereof of claims 1-5 prior to transplantation of the organ to the recipient or within a day of the transplantation,
thereby reducing delayed graft function following organ transplantation.

25. The method of claim 24, wherein the organ is a kidney, a heart, or a lung.

26. A method of treating an organ prior to transplantation into a recipient, comprising contacting the organ with a composition comprising an antibody or antigen-binding fragment thereof of claims 1-5.

27. The method of claim 26, wherein the organ is a kidney, a heart, or a lung.

28. The method of claim 26, wherein the contacting step comprises perfusing the organ with the composition comprising a polypeptide or antibody that immunospecifically binds the epitope recognized by mab107.

29. A method of treating a subject having an autoimmune disease comprising administering to the subject a therapeutically effective amount of composition comprising an antibody or antigen-binding fragment thereof of claims 1-5.

30. The method of claim 29, wherein the autoimmune disease is cytoplasmic antineutrophil cytoplasmic antibodies (cANCA)-associated vasculitis.

31. A method of treating a subject having diabetic nephropathy comprising administering to the subject a therapeutically effective amount of composition comprising an antibody or antigen-binding fragment thereof of claims 1-5.

32. A method of ameliorating a pathology associated with chemotherapy in a subject comprising administering to the subject a therapeutically effective amount of composition comprising an antibody or antigen-binding fragment thereof of claims 1-5.

33. The method of claim 32, wherein the pathology is Adriamycin nephropathy.

34. A composition comprising an antibody or antigen-binding fragment thereof of claims 1-5, and a pharmaceutically acceptable carrier.

35. A nucleic acid molecule encoding a humanized mAb107 antibody or antigen-binding fragment of any one of claims 1-5

36. A recombinant vector comprising the nucleic acid molecule of claim 35.

37. A host cell comprising the recombinant vector of claim 36 and or the nucleic acid molecule of claim 35.

38. The host cell of claim 37, wherein the host cell is selected from the group consisting of E. coli, P. pastoris, Sf9, COS, HEK293, and CHO.

39. A pharmaceutical composition comprising:
(a) one or more of the nucleic acid molecule of claim 35, the recombinant vector of claim 36, or the host cell of claims 37-38; and
(b) a pharmaceutically acceptable carrier.


SEQUENCE LISTING (18 sequences)

SEQ ID NO: 1 - protein, 122 aa, Artificial
  Humanized Heavy Chain mAb107
QVQLVQSGAEVKKPGASVKVSCKPSGFNIKDIYMQWVRQAPGQRLEWIGRIDPADDKTKYDPKFQGRATITADTSASTAYLELSSLRSEDTAVYYCASEGHYGYDGYAMDYWGQGTTVTVSS

SEQ ID NO: 2 - protein, 113 aa, Artificial
  Humanized Light Chain mAb107
DIVMTQSPDSLAVSLGERATINCKSSQNLLYSSNQKNYLAWYQQKPGQPPKLLIYWASTRESGVPDRFSGSGSGTDFTLTISSLQAEDVAVYYCQQYYSYPLTFGQGTKLEIK

SEQ ID NO: 3 - protein, 218 aa, Artificial
  Humanized Heavy Chain mAb107 IgG4kappa
QVQLVQSGAEVKKPGASVKVSCKPSGFNIKDIYMQWVRQAPGQRLEWIGRIDPADDKTKYDPKFQGRATITADTSASTAYLELSSLRSEDTAVYYCASEGHYGYDGYAMDYWGQGTTVTVSSASTKGPSVFPLAPCSRSTSESTAALGCLVKDYFPEPVTVSWNSGALTSGVHTFPAVLQSSGLYSLSSVVTPSSSLGTKTYTCNVDHKPSNTKVDKR

SEQ ID NO: 4 - protein, 220 aa, Artificial
  Humanized Light Chain mAb107 IgG4kappa
DIVMTQSPDSLAVSLGERATINCKSSQNLLYSSNQKNYLAWYQQKPGQPPKLLIYWASTRESGVPDRFSGSGSGTDFTLTISSLQAEDVAVYYCQQYYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 6 aa, Artificial
  CDR1 of the VH of SEQ ID NO 1
GFNIKD

SEQ ID NO: 6 - protein, 7 aa, Artificial
  CDR2 of the VH of SEQ ID NO1
PADDKTK

SEQ ID NO: 7 - protein, 9 aa, Artificial
  CDR3 of the VH of SEQ ID NO 1
GHYGYDGYA

SEQ ID NO: 8 - protein, 13 aa, Artificial
  CDR1 of the VL of SEQ ID NO 2
SQNLLYSSNQKNY

SEQ ID NO: 9 - protein, 12 aa, Artificial
  CDR2 of the VL of SEQ ID NO 2
WASTRESGVPDR

SEQ ID NO: 10 - protein, 6 aa, Artificial
  CDR3 of the VL of SEQ ID NO 2
YYSYPL

SEQ ID NO: 11 - DNA, 1413 bp, Artificial
  Humanized mutagenized mAB 107 IgG4kappa heavy chain V5
atggagacagacacactcctgctatgggtactgctgctctgggttccaggttccactggtgaccaggtgcagctggtgcagagcggcgcggaagtgaagaaaccgggcgcgagcgtgaaagtgagctgcaagccgagcggctttaacattaaagatatttatatgcagtgggtgaggcaggcgccgggccagcgcctggaatggattggccgcattgatccggcggacgataaaaccaaatatgatccgaaatttcagggccgcgcgaccattaccgcggataccagcgcgagcaccgcgtatctggaactgagcagcctgcgcagcgaagataccgcggtgtattattgcgcgagcgaaggccattatggctatgatggctatgcgatggattattggggccagggcaccaccgtgaccgtgagcagcgctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 12 - protein, 470 aa, Artificial
  Humanized mutagenized mAB 107 IgG4kappa heavy chain V5
METDTLLLWVLLLWVPGSTGDQVQLVQSGAEVKKPGASVKVSCKPSGFNIKDIYMQWVRQAPGQRLEWIGRIDPADDKTKYDPKFQGRATITADTSASTAYLELSSLRSEDTAVYYCASEGHYGYDGYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Artificial
  Leader sequence
METDTLLLWVLLLWVPGSTGD

SEQ ID NO: 14 - DNA, 726 bp, Artificial
  Humanized mAb107 IgG4 kappa Light Chain
atggagacagacacactcctgctatgggtactgctgctctgggttccaggttccactggtgacgatattgtgatgacccagagcccggatagcctggcggtgagcctgggcgaacgcgcgaccattaactgcaaaagcagccagaacctgctgtatagcagcaaccagaaaaactatctggcgtggtatcagcagaaaccgggccagccgccgaaactgctgatttattgggcgagcacccgcgaaagcggcgtgccggatcgctttagcggcagcggcagcggcaccgattttaccctgaccattagcagcctgcaggcggaagatgtggcggtgtattattgccagcagtattatagctatccgctgacctttggccagggcaccaaactggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcacccgtcacaaagagcttcaacaggggagagtgttag

SEQ ID NO: 15 - protein, 260 aa, Artificial
  scFv of humanized mutated mAb107
EFQVQLVQSGAEVKKPGASVKVSCKPSGFNIKDIYMQWVRQAPGQRLEWIGRIDPADDKTKYDPKFQGRATITADTSASTAYLELSSLRSEDTAVYYCASEGHYGYDGYAMDYWGQGTTVTVSSGGGGSGGGGSGGGGSDIVMTQSPDSLAVSLGERATINCKSSQNLLYSSNQKNYLAWYQQKPGQPPKLLIYWASTRESGVPDRFSGSGSGTDFTLTISSLQAEDVAVYYCQQYYSYPLTFGQGTKLEIKENLYFQGS

SEQ ID NO: 16 - protein, 15 aa, Artificial
  GS Linker
GGGGSGGGGSGGGGS

SEQ ID NO: 17 - protein, 225 aa, Artificial
  mAb107 IgG1
QVQLVQSGAEVKKPGASVKVSCKPSGFNIKDIYMQWVRQAPGQRLEWIGRIDPADDKTKYDPKFQGRATITADTSASTAYLELSSLRSEDTAVYYCASEGHYGYDGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 241 aa, Artificial
  Humanized Light Chain mAb107 IgG4kappa with leader sequence
METDTLLLWVLLLWVPGSTGDDIVMTQSPDSLAVSLGERATINCKSSQNLLYSSNQKNYLAWYQQKPGQPPKLLIYWASTRESGVPDRFSGSGSGTDFTLTISSLQAEDVAVYYCQQYYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC